(19) United States
(12) Patent Application Publication
(10) Pub. No.: US 2007/0141027 A1
(43) Pub. Date: Jun. 21, 2007

(54) Non-lethal conditioning methods for conditioning a recipient for bone marrow transplantation

(75) Inventor: Suzanne T. Ildstad, Prospect, KY (US)

Correspondence Address: FISH & RICHARDSON P.C., PO BOX 1022, MINNEAPOLIS, MN, 55440-1022, US

(73) Assignee: UNIVERSITY OF LOUISVILLE RESEARCH FOUNDATION INC., Louisville, KY (US)

(21) Appl. No.: 10/558,513

(22) PCT Filed: May 28, 2004
(86) PCT No.: PCT/US04/17051

§ 371 Date: Nov. 13, 2006

Related U.S. Application Data
(60) Provisional application No. 60/473,791, filed on May 28, 2003.

(51) Int. Cl. A61K 39/395; A61K 48/00

(52) U.S. Cl. 424/93.2; 424/144.1

(57) ABSTRACT

Mixed chimerism induces donor-specific transplantation tolerance to organ allografts. Strategies to establish mixed chimerism using partial conditioning having significantly reduced the morbidity associated with conditioning. The donor hematopoietic cell lineage(s) responsible for the induction and subsequent maintenance of tolerance in partially conditioned recipients are not defined at present. As one approaches the threshold for nonmyeloablative conditioning, donor factors that influence the induction of tolerance have become apparent. In this invention, recipient B10 (H2^{b}) mice were pretreated in vivo with anti-αβ-TCR and anti-CD8 mAbs 3 days before TBI (day 0) and transplanted with 15×10⁶ allogeneic (B10.BR;H2^{k}) marrow cells. Engraftment occurred in 20%, 75% and 94% of animals conditioned with 100, 200 or 300 cGy TBI once month post BMT, respectively. In those animals that engrafted some exhibited multilineage production, including donor T cells, while others had only donor B cell, NK cell, macrophage, granulocyte and dendritic cell production. Animals without donor T cells lost their chimerism gradually within 6 months and rejected both donor and third-party skin grafts, even when they had significant (up to 70%) levels of donor chimerism. In animals with donor T cell production, chimerism remained stable for >_6 months and donor skin grafts were accepted. In the animals without donor T cell production, none of the expected stages of T cell development were present in the thymus, while in those with donor T cell production they were. Moreover, clonal deletion of Vβ 5.1/2⁺ and Vβ 11⁺ CD8 and CD4 T cells occurred only in chimeras with donor T cell production. These results show for the first time that donor T cell production plays a critical role in the maintenance of durable chimerism and induction of transplantation tolerance, and is directly correlated with deletion of potentially autoreactive cells.

### CROSS-REFERENCE TO RELATED APPLICATIONS

This application claims priority of U.S. Provisional Application Ser. No. 60/473,791 filed May 28, 2003, which is incorporated herein by this reference in its entirety.

### CONTRACTUAL ORIGIN OF THE INVENTION

This invention was supported in part by NIH Grant No. DK43901-07, awarded by the National Institutes of Health. The government has certain rights to this invention.

### BACKGROUND OF THE INVENTION

1. Field of the Invention

The present invention pertains to the effect of pretreatment of a bone marrow recipient in vivo with anti-αβ-TCR and anti-CD8 mAbs 3 days before TBI (Total Body Irradiation) (day 0) on the minimum effective TBI dose needed and the durability of chimerism and correlation with tolerance. The donor specific lineage production in mixed chimeras were monitored and examined for their role in allograft tolerance induction. The invention disclosed herein provides methods for pretreatment of the recipient with anti-αβ-TCR and anti-CD8 mAbs, which reduces the TBI requirement for establishing mixed chimerism. Because chimerism remains stable only in those chimeras with donor T cell engraftment, the methods of the present invention underscore the important role for donor T cells in the early stages of engraftment. Donor-specific tolerance is observed only in chimeras with donor T cell production. Accordingly, the present invention provides a role for donor T cells in tolerance induction.

2. Description of the State of Art

The transfer of living cells, tissues, or organs from a donor to a recipient, with the intention of maintaining the functional integrity of the transplanted material in the recipient defines transplantation. Transplants are categorized by site and genetic relationship between the donor and recipient. An autograft is the transfer of one's own tissue from one location to another; a syngeneic graft (isograft) is a graft between identical twins; an allogeneic graft (homograft) is a graft between genetically dissimilar members of the same species; and a xenogeneic graft (heterograft) is a transplant between members of different species.

A major goal in solid organ transplantation is the permanent engraftment of the donor organ without a graft rejection immune response generated by the recipient, while preserving the immunocompetence of the recipient against other foreign antigens. Typically, in order to prevent host rejection responses, nonspecific immunosuppressive agents such as cyclosporine, methotrexate, steroids and FK506 are used. These agents must be administered on a daily basis and, if stopped, graft rejection usually results. However, a major problem in using nonspecific immunosuppressive agents is that they function by suppressing all aspects of the immune response, thereby greatly increasing a recipient's susceptibility to opportunistic infections, rate of malignancy, and end-organ toxicity. The side effects associated with the use of these drugs include opportunistic infection, an increased rate of malignancy, and end-organ toxicity (Dunn, D. L., Crit. Care. Clin., 6:955 (1990)). Although immunosuppression prevents acute rejection, chronic rejection remains the primary cause of late graft loss (Nagano, H., et al., Am J. Med. Sci, 313:305-309 (1997)).

For every organ, there is a fixed rate of graft loss per annum. The five-year graft survival for kidney transplants is 74% (Terasaki, P. I., et al., UCLA Tissue Typing Laboratory (1992)). Only 69% of pancreatic grafts, 68% of cardiac transplants and 43% of pulmonary transplants function 5 years after transplantation (Opelz, G., Transplant Proc, 31:31S-33S (1999)). The only known clinical condition in which complete systemic donor-specific transplantation tolerance occurs is when chimerism is created through bone marrow transplantation (Qin, et al., J. Exp. Med., 169:779 (1989); Sykes, et al., Immunol. Today, 9:23-27 (1988); and Sharabi, et al., J. Exp. Med., 169:493-502 (1989)). This has been achieved in neonatal and adult animal models as well as in humans by total lymphoid or body irradiation of a recipient followed by bone marrow transplantation with donor cells.

The success rate of allogeneic bone marrow transplantation is, in large part, dependent on the ability to closely match the major histocompatibility complex (MHC) of the donor cells with that of the recipient cells to minimize the antigenic differences between the donor and the recipient, thereby reducing the frequency of host-versus-graft responses and graft-versus-host disease (GVHD). In fact, MHC matching is essential, only a one or two antigen mismatch is acceptable because GVHD is very severe in cases of greater disparities. Thus, MHC plays a vital role in tumor or skin transplantation and immune responsiveness.

A major histocompatibility complex (MHC) is present in all vertebrates, and the mouse MHC (commonly referred to as H-2 complex) and the human MHC (commonly referred to as the Human Leukocyte Antigen or HLA) are the best characterized. The MHC is a cluster of closely linked genetic loci encoding three different classes (class I, class II, and class ml) of glycoproteins expressed on the surface of both donor and host cells that are the major targets of transplantation rejection immune responses. The MHC is divided into a series of regions or subregions and each region contains multiple loci. Different loci of the MHC encode two general types of antigens which are class I and class II antigens. In the mouse, the MHC consists of 8 genetic loci: Class I is comprised of K and D, class II is comprised of I-A and/or I-E. The class II molecules are each heterodimers, comprised of I-Aα and I-Aβ and/or I-Eα and I-Eβ.

The major function of the MHC molecule is immune recognition by the binding of peptides and the interaction with T cells, usually via the αβ T-cell receptor. It was shown that the MHC molecules influence graft rejection mediated by T cells (Curr. Opin. Immunol., 3:715 (1991), as well as by NK cells (Annu. Rev. Immunol., 10:189 (1992); J. Exp. Med., 168:1469 (1988); Science, 246:666 (1989). The induction of donor-specific tolerance by HSC chimerism overcomes the requirement for chronic immunosuppression. (Ildstad, S. T., et al., Nature, 307:168-170 (1984), Sykes, M., et al., Immunology Today, 9:23-27 (1998), Spitzer, T. R., et al., Transplantation, 68:480-484 (1999)). Moreover, bone marrow chimerism also prevents chronic rejection (Colson, Y., et al., Transplantation, 60:971-980 (1995); and Gammie, J. S., et al., In Press Circulation (1998)). The association between chimerism and tolerance has been demonstrated in numerous animal models including rodents, (Ildstad, S. T., et al., Nature, 307:168-170 (1984); and Billingham, R. E., et al., Nature, 172:606 (1953)) large animals, primates and humans (Knobler, H. Y., et al., Transplantation, 40:223-225 (1985); Sayegh, M. H., et al., Annals of Internal Medicine, 114:954-955 (1991)).

As mentioned previously, bone marrow transplantation is the only known method to achieve systemic tolerance of organ transplantation. Bone marrow, a spongy tissue found in the cavities of bones, contains hematopoietic stem cells (HSC). Each type of blood cell begins its life as an HSC. The HSC divide and differentiate to form the various cells found in blood and immune systems, including leukocytes, lymphocytes, erythrocytes and platelets. During this procedure, some HSC retain a long-term multilineage repopulating potential (self-renewal); and some HSC may only retain a short-term multilineage repopulating potential and differentiate to produce progeny.

The major purified HSC transplantation-related complications include graft rejection and graft failure. The outcome for engraftment of highly purified HSC in the major histocompatibility complex (MHC)-matched recipients is different from that for MHC-disparate allogeneic recipients (El-Badri, N. S., Good, R. A., (1993); and Kaufman, C. L., S. Cell Biochem Suppl., 18:A112 (1994)).

The hematopoietic microenvironment also plays a major role in the transplantation and engraftment of HSC. For example, the microenvironment is a source of growth factors and cellular interactions for the survival and renewal of HSC. A number of cell types collectively referred to as stromal cells are found in the vicinity of the HSC in the bone marrow microenvironment These stromal cells include both bone marrow-derived CD45⁺ cells and non-bone marrow-derived CD45⁻ cells, such as adventitial cells, reticular cells, endothelial cells and adipocytes. Recently, Ildstad, et al., identified another cell type known as hematopoietic facilitatory cells, which when co-administered with donor bone marrow cells enhance the ability of the donor cells to stably engraft in allogeneic and xenogeneic recipients. See U.S. Pat. No. 5,772,994, which is incorporated herein by reference.

The facilitatory cells and the stromal cells occupy a substantial amount of space in a recipient's bone marrow microenvironment, which may appear to present a barrier to donor cell engraftment. However, it has been shown that HSC bind to facilitatory cells in vitro and in viva. Thus, contrary to the stromal cells, the facilitatory cells may provide physical space or niche on which the stem cells survive and are nurtured. It is therefore believed to be desirable to develop conditioning regimens to specifically target and eliminate the stromal cell populations in order to provide the space necessary for the HSC and the associated facilitatory cells. Furthermore, it is believed that such conditioning regimens may facilitate a donor cell preparation to engraft without the use of lethal irradiation. See U.S. Pat. Nos. 5,635,156 and 5,876,692 which are incorporated herein by reference.

Until fairly recently, it was believed by those skilled in the art that lethal conditioning of a human recipient, which renders the recipient totally immunocompetent, was required to achieve successful engraftment of donor bone marrow cells in the recipient. Now, a number of sublethal conditioning approaches using less aggressive cytoreduction have been reported in rodent models (Mayumi and Good, J. Exp. Med., 169:213 (1989); Slavin, et al., J. Exp. Med., 147(3):700 (1978); McCarthy, et al., Transplantation, 40(1):12 (1985); Sharabi, et al., J. Exp. Med., 172(1):195 (1990); and Monaco et al., Ann. NY Acad. Sci, 129:190 (1966)). Sublethal conditioning renders the recipient only partially immunocompetent. However, these sublethal conditioning approaches have not demonstrated reliable and stable donor cell engraftment, and long-term tolerance has remained a question in many of these models (Sharabi and Sachs, J. Exp. Med., 169:493 (1989); Cobbold, et al., Immunol. Rev., 129:165 (1992); and Qin, et al., Eur. J. Immunol., 20:2737 (1990)). Also, reproducible engraftment has not been achieved, especially when multimajor and multiminor antigenic disparities existed.

In any event, whether lethal or sublethal, irradiation of the host poses a significant limitation to the potential clinical application of bone marrow transplantation to a variety of disease conditions where suppression of the immune system is undesirable, including solid organ or cellular transplantation, sickle cell anemia, thalassemia and aplastic anemia. Accordingly, other methods to induce tolerance of donor bone marrow cells have been investigated.

Early work by Wood and Monaco attempted to induce tolerance using bone marrow plus anti-lymphocyte serum (ALS) in partial MHC-matched donor-recipient combinations (Wood, et al., Trans. Proc., 3(1):676 (1971); Wood and Monaco, Transplantation, (Baltimore) 23:78 (1977)). Even in this semi-allogeneic system, F, splenocytes were required to facilitate the induction of tolerance, and thymectomy was required for stable long-term tolerance. The additional requirement for splenocytes and thymectomy made potential clinical applicability of such an approach unlikely. However, these studies identified two key factors required for induction of tolerance: an antigenic source of tolerogen, which is not only involved in tolerance induction, but must also be present at least periodically for permanent antigen-specific tolerance, and a method to tolerize or prevent activation of new T cells from the thymus, i.e. thymectomy, or intrathymic clonal deletion.

Attempts to induce tolerance to allogeneic bone marrow donor cells using combinations of depleting and non-depleting anti-CD4 and CD8 monoclonal antibodies (mAb) resulted in only transient tolerance to MHC-compatible combinations (Cobbold, et al., Immunol Rev, 129:165 (1992); and Qin, et al., Eur. J. Immunol., 20:2737 (1990)). 6Gy of TBI was required to obtain stable engraftment and tolerance when MHC-disparate bone marrow was utilized (Cobbold, et al., Transplantation, 42:239 (1986)). Sharabi and Sachs attributed the failure of anti-CD4/CD8 mAb therapy alone to the inability of mAb to deplete T cells from the thymus, since persistent cells coated with mAb could be identified in this location (Sharabi and Sachs, J. Exp. Med., 0.169:493-502 (1989)). However, subsequent attempts to induce tolerance by the addition of 7Gy of selective thymic irradiation prior to donor bone marrow transplantation also failed. Engraftment was only achieved with the addition of 3Gy of recipient TBI.

The administration of a combination of mAb plus chemotherapeutic agents such as cyclophosphamide has also been successful in achieving engraftment in closely matched donor and recipient combinations (Mayumi, H., et al., Transplantation Proceedings, 20:139-141 (1998)).

Yet, when greater genetic disparities exist between donor and recipient, more conditioning is required to achieve engraftment. The level of risk inherent in tolerance-inducing conditioning approaches must be balanced against the disorder. When the disorder is morbid, but relatively benign, or in cases of solid organ transplantation, hematologic disorders, including aplastic anemia, severe combined immunodeficiency (SCID) states, thalassemia, diabetes and other autoimmune disease states, sickle cell anemia, and some enzyme deficiency states, a nonlethal preparative regimen, which would allow partial engraftment of allogeneic or even xenogeneic bone marrow to create a mixed host/donor chimeric state is preferred. For example, it is known that only approximately 40% of normal erythrocytes are required to prevent an acute sickle cell crisis (Jandle, et al., Blood, 18(2) (1961); Cohen, et al., Blood, 18(2):133 (1961); and Cohen, et al., Blood, 76(7) (1984)), making sickle cell disease a prime candidate for an approach to achieve mixed multilineage chimerism.

Attempts to achieve engraftment and tolerance in MHC-mismatched combinations have not enjoyed the same success as the aforementioned methods used in MHC-compatible combinations. In most models, only transient donor-specific tolerance has been achieved (Mayumi, et al., Transplantation, 44(2):286 (1987); Mayumi, et al., Transplantation, 42(4):286 (1986); Cobbold, et al., Eur. J. Immunol., 20:2747 (1990); and Cobbold, et al., Seminars in Immunology, 2:377 (1990)).

For example, in microchimerism models, in spite of the presence of microchimerism, most solid organ allograft recipients do not become drug free, apparently due to a dissociation of the presence of microchimerism and the establishment of functional tolerance in solid organ allograft recipients. Although low levels of donor cells might be detected systemically in recipients of heart and liver allografts, donor specific tolerance is not generally associated with the microchimeric state. Others have analyzed a cohort of transplant patients and showed a similar frequency and severity of rejection episodes in patients with and without microchimerism as defined by nested PCR technique (Elwood, et al., Lancet, 349:1358 (1997)). In a swine model, swine leukocyte Ag (SLA)-identical pigs underwent a renal allograft coincident with a 12-day course of high-dose cyclosporine. Although the pigs accepted their grafts, the maintenance of tolerance to kidney allografts did not result in the persistence of donor cell microchimerism (Fuchimoto, et al., J. Immunol., 162:5704 (1999)). It has therefore been argued that microchimerism is a result, but not a cause, of long-term graft survival (Monaco, et al., Transplant. Proc., 33:3837 (2001)).

Similarly, in macrochimerism models, the dissociation of chimerism and allogeneic tolerance has also been observed. In an allogeneic mouse model similar to that used in this study, a small fraction of animals conditioned with anti-CD8 and anti-CD4 mAb plus 300 cGy TBI failed to produce significant numbers of donor-type T cells and the donor chimerism declined quickly. Recipients with this chimeric profile donor-specific skin grafts (Tomita, et al., J. Immunol., 153:1097 (1994)). A dissociation of chimerism and tolerance has also been observed in animal models in which T cell split chimerism is established using different T cell KO mice as donors. When knockout mice deficient in both CD4 and CD8 T cells or CD36-transgenic mice lacking both T cells and natural killer (NK) cells were used as donors, high levels of donor chimerism resulted but the animals were not tolerant to donor-specific grafts (Umemura, et al, J. Immunol., 167:3043 (2001)).

Engraftment across MHC barriers has been achieved with low dose irradiation in combination with pre-treatment of the host with depleting and nondepleting CD4 and CD8 mAbs, (Cobbold, S. P., et al., Nature, 328:164-166 (1986)), or the use of mAbs in combination with thymic irradiation (Sharabi, Y., et al, J. Experimental Medicine, 169:493-502 (1989)). In MHC plus minor antigen disparate mice conditioned with 1 mg ALG on day −3 and 200 mg/kg cyclophosphamide on day +2 and transplanted with 15×10⁶ allogeneic bone marrow cells, durable multi-lineage chimerism occurs with as low as 200 cGy total body irradiation (Colson, Y. L., et al., J. Immunology, 157:2820-2829 (1996)). When 30×10⁶ bone marrow cells are transplanted, engraftment can be achieved with 100 cGy TBI in this model (Colson, Y. L., J. Immunology, 157:2820-2829 (1996)). Replacement of ALG with in vivo administration of anti-CD4 and anti-CD8 antibodies results in engraftment in 100% of recipients, and the level of chimerism is actually higher (Exner, B. G., et al., Surgery, 122:211-227 (1997)). Moreover, anti-CD8 mAb alone is more efficient at ensuring engraftment than ALG (Exner, B. G., et al., Surgery, 122:211-227 (1997)).

In nonradiation-based protocols using ALS and rapamycin, it has been clearly shown that T cells are not required in the BM for tolerance induction (Hale, et al., Transplantation, 69:1242 (2000)). In these studies, C57BL/10 recipients received ALS (days −1 and +2) relative to B10.A skin grafts (day 0), sirolimus (day 6), and megadoses of BMC on day 7. Allogeneic chimerism was achieved, but donor T cells were not produced. Interestingly, chimeras showed specific tolerance, as evidenced by acceptance of second-donor grafts and rejection of third-party grafts. The role of donor T cells for tolerance induction in this model was also examined using knockout mice as donors (Umemura, et al, Transplantation, 70:1005 (2000)). BM from mice lacking CD4, CD8α, CD4 plus CD8α, or CD3ε expressing cells was as effective in inducing tolerance as wild-type BM. The explanation for the differences of donor T cell chimerism in its role of tolerance induction between different conditioning regimens is not known. Furthermore, in this nonradiation-based model, donor class II antigen-positive BM cells were required for tolerance induction (Umemura, et al, J. Immunol., 164:4452 (2000)), and when class II deficient KO mice were utilized as donors, tolerance did not occur.

Taken together these data suggest that the donor hematopoietic cells for tolerance induction might be influenced by the conditioning approach used. The caveat to these studies is that mAb conditioning of the recipient in vivo may not fully remove the effector cell population targeted, resulting in graft rejection.

T-cells have been implicated as the primary effector cells in solid organ allograft rejection. Eto, et al., described that targeting αβ-TCR⁺ T-cells significantly prolonged survival of skin grafts. While the same effect could be achieved by targeting CD3⁺ T-cells, animals prepared by depletion of αβ-TCR⁺ cells demonstrated relatively superior immunocompetence (Eto, M., et al., et al., Immunology, 81:198-204 (1994)). A critical and non-redundant role for host αβ-TCR⁺ T-cells as effector cells in the rejection of heart allografts, since TCR-β KO mice did not reject MHC-disparate cardiac allografts was recently reported (Exner, B. G., et al., Surgery, 126:121-126 (1999)). Bone marrow transplantation (BMT) from normal B10.BR donors restored the immunocompetence to reject third-patty cardiac allografts in TCR-β KO mice (Exner, B. G., et al., Surgery, 126:121-126 (1999)).

T-cells also play an important role in the rejection of bone marrow grafts. When Kernan, et al., characterized the cells present in recipients of HLA-mismatched bone marrow grafts at the time of rejection, they found that graft failure was associated with the emergence of donor-reactive T-cells. (Kernan, N. A., et al., Transplantation, 43:842-847 (1987)). Other groups describe that CD2⁺, CD3⁺ and CD8⁺ T-cells of recipient origin in the peripheral blood of bone marrow recipients effectively inhibit the proliferation and differentiation of donor bone marrow cells in vitro (Bierer, B. E., et al., Transplantation, 46:835-839 (1988)).

As discussed in detail above, conditioning of the recipient with a combination of cytoreductive plus immunosuppressive agents has been required to achieve engraftment of MHC-disparate marrow (Colson, Y. L., et al., J. Immunology, 157:2820-2829 (1997); Gamm, J. S., et al., Experimental Hematology, 26:927-935 (1998)). For the most part, the approach to cytoreduction has involved nonspecific immunosuppressive agents, such as irradiation and busulfan, which have a broad specificity and poorly defined mechanism of action. If those specific host components that regulate engraftment could be defined, more specific approaches to target only those specific host cells responsible for alloresistance to engraftment would be possible.

Therefore, there remains a need for non-lethal methods of conditioning a recipient for allogeneic bone marrow transplantation that would result in stable mixed multilineage allogeneic chimerism and long term-donor-specific tolerance.

### SUMMARY OF THE INVENTION

The present invention provides methods for conditioning a recipient for bone marrow transplantation. The conditioning method of the present invention utilizes a composition that specifically depletes αβ-TCR⁺ T cells and CD8⁺ T cells in the recipient hematopoietic microenvironment.

In one embodiment of this aspect of the invention, the composition comprises antibodies specific for αβ-TCR⁺ T cells and CD8⁺ T cells are used to target and deplete such cells in the recipient.

In another embodiment of this aspect of the invention, the composition comprises antisense DNA and is directed against the precursors of αβ-TCR⁺ T cells and CD8⁺ T cells. Alternatively, the antisense DNA alters the translation or transcription of the αβ-TCR⁺ T cells and CD8⁺ T cells.

In yet another embodiment, the composition utilized in the methods of the present invention comprises a cytotoxic drug specific for αβ-TCR⁺ T cells and CD8⁺ T cells.

In still another aspect, the methods of the present invention further contemplate subjecting the recipient to further conditioning by total body irradiation or an alkylating agent.

The present invention further contemplates providing a method of partially or completely reconstituting a mammal's lymphohematopoietic system comprising administering to the mammal a composition that specifically depletes αβ-TCR⁺ T cells and CD8⁺ T cells in the recipient hematopoietic microenvironment.

### BRIEF DESCRIPTION OF THE DRAWINGS

The accompanying drawings, which are incorporated in and form a part of the specification, illustrate the preferred embodiment of the present invention, and together with the description serve to explain the principles of the invention.

In the Drawings:

FIG. 1. Characteristics of engraftment and the level of donor chimerism in B10 recipients conditioned with increasing dose of TBI. B10 mice were pretreated with anti-αβ-TCR and anti-CD8 mAbs on day −3. On day 0, they were transplanted with 15×10⁶ untreated bone marrow cells from B10.BR donors 4-6 hours after conditioning with 100, 200 or 300 cGy TBI. Unconditioned controls were also performed. Animals were analyzed for engraftment by flow cytometric analysis monthly for up to 6 months after BMT (n=number of animals in each experiment). FIG. 1 shows the frequency of engraftment at 1 month after BMT (A), level of chimerism in animals that engrafted (percentage of donor cells in PBL) at 1 month (B), and kinetics of engraftment for up to 0.6 months after BMT (C) as assessed by PBL typing. The results are the summary of 3 experiments.

FIG. 2. Detection of donor and host derived cells of lymphoid and myeloid lineages in mixed allogeneic chimeras using two-color flow cytometry. Multilineage typing was performed between 2 to 3 months post BMT on animals that exhibited high levels of donor chimerism. The x axis shows staining with fluorescein-conjugated antibody against donor class I antigen (H21a). On the y axis the staining for the different lineages is shown: αβ-TCR, CD8, CD4, NK1.1 (NK cells), B220 (B cells), Mac-1 (macrophages), Gr-1 (granulocytes) and CD11c (dendritic, cells). Results of two representative chimeras are presented. FIG. 2A: Donor-derived B-cells, NK cells, monocytes and dendritic cells were detected in these mixed chimeras. Donor T cells were not produced. FIG. 2B: All lineages of donor origin were present in these chimeras. Moreover, all lineages of host origin were evaluated in both groups of chimeras.

FIG. 3. Production of donor T cells is critical for the maintenance of stable chimerism. Animals were analyzed for the level of donor chimerism by flow cytometric analysis monthly for up to 6 months after BMT. Animals rendered chimeric by preconditioning with αCD8 plus αβ-TCR mAb and varying doses of TBI were divided into two groups according the results from PBL typing for multilineage engraftment: 1) chimeras without donor T-cell engraftment; and 2) chimeras with donor T cell engraftment. FIGS. 3A and 3B show the kinetics of the level of donor chimerism in each individual animal for up to 6 months after BMT in groups of chimeras without (FIG. 3A) and with (FIG. 3B) donor T cell engraftment. The results are a summary of 4 experiments.

FIG. 4. Production of donor T cells is critical for the induction of donor-specific tolerance to skin grafts. Animals were divided into 3 three groups according to the results at 1 month from PBL typing for donor chimerism and multilineage engraftment: 1) animals without engraftment; 2) chimeras without donor T cell engraftment; and 3) chimeras with donor T cell engraftment. Each animal received skin grafts from donor-specific (B10.BR) and third-party (BALB/c, H2^{d}) strains 2-3 months after BMT and the grafts were then monitored up to 120 days. ^{a}Level of donor chimerism at the time when skin transplantation was performed. ^{b}Median survival time of skin grafts±standard deviation. Donor specific skin graft survival in the group with donor T cell engraftment was significantly longer than in the group without donor T cell engraftment (P<0.00005).

FIG. 5: One-way MLR assay. Lymphocytes from mixed chimeras with (n=8) or without (n=6) peripheral donor T cells (PDTC) as well as from recipients that did not engraft (non-chimeras, n=7) were co-cultured with irradiated host (B10), donor (B10.BR) and third-party (BALB/c) stimulator cells in MLR assay. Values are shown as mean±SD of triplicate cultures in a 1:1 responder to stimulator ratio from a representative sample.

FIG. 6. Relative Vβ-TCR expression in chimeras with or without donor T cell production. Expression of Vβ 5.1/2 (open bars), Vβ 6 (dotted bars), Vβ 8.1/2 (hatched bars), and Vβ 11 (filled bars) in unmanipulated hosts (1310), unmanipulated donors (B10.BR), chimeras with donor T cell engraftment or chimeras without donor T cell engraftment were measured by FACS analysis. Relative expression represents the percentage of Vβ-positive cells within the CD8⁺ (FIG. 6A) or CD4⁺ FIG. 6B) T cell subsets of the host (H2K^{b}) lymphogate in peripheral blood. Data from three experiments are depicted as mean±SD. Vβ-TCR expression in either chimeric group was compared with that in B10 mice using one tailed t-test (two sample assuming unequal variances). Significant P values are indicated above the respective data bars (*P≦0.005 and ^{Ψ}P≦0.05).

FIG. 7. Analysis of the expression of CD24 in CD4⁻/8⁻ thymocytes in chimeras with or without donor T cells in PB. Thymocytes were prepared from chimeras with (n=5) or without (n=4) peripheral donor T cells (PDTC) and from naïve control B10 or B10.BR mice. They were stained with CD4 APC, CD8 PE, CD24 PerCP and donor class I H2-K^{k} FITC. Cells that were negative for both CD4 and CD8 were gated (A) and further analyzed for the expression of donor class I and CD24 (FIG. 7B). Data shown is representative staining of one mouse from each group.

FIG. 8. Engraftment (%) in mice pretreated with indicated mAb or mAb combinations and conditioned with 300 cGy. Recipient B10 mice were pretreated with the indicated mAb or mAb combinations of mAbs 3 days before BMT to target different T cell populations. On day 0, they were transplanted with untreated 15×10⁶ bone marrow cells from B10.BR donors 4-6 hours after conditioning with 300 cGy TBI. Animals were tested for chimerism by flow cytometric analysis monthly for up to 6 months after BMT. n=number of animals in each experiment.

FIG. 9. Donor multilineage engraftment in recipients with or without donor T cell engraftment. Multilineage typing was performed in mice between 2 to 3 months post BMT.

### DETAILED DESCRIPTION OF THE INVENTION

The methods of the present invention are to be administered for the purpose of conditioning a recipient for bone marrow transplantation. The conditioning methods of the present invention comprise various compositions that specifically deplete up-TCR⁺ T cells and CD8⁺ T cells in the recipient hematopoietic microenvironment. The conditioning methods of the present invention are followed by transplantation with a donor cell preparation containing hematopoietic stem cells from a donor that are matched at the major histocompatibility complex class I K locus with the recipient hematopoietic microenvironment.

Hematopoietic stem cell (HSC) chimerism induces donor-specific tolerance to solid organ grafts. The inventor previously demonstrated that partial conditioning with 700 cGy total body irradiation (TBI) was sufficient to achieve engraftment of major histocompatibility complex (MHC)-disparate allogeneic mouse marrow in 100% of recipients. The effective TBI dose could be significantly reduced if anti-lymphocyte globulin, T-cell-specific mAbs, cytotoxic drugs, or costimulation blocking agents were added. As the minimum threshold of conditioning to establish and maintain tolerance is approached, a dissociation between chimerism and tolerance has emerged which allow an understanding of the early events which influence the induction of tolerance.

Mixed hematopoietic chimerism induces tolerance to solid organ and cellular grafts and has the following advantages: 1) superior immunocompetence; 2) avoidance of chronic rejection; and 3) establishment with nonmyeloablative conditioning, thereby reducing the mortality associated with ablative conditioning. A number of incompletely ablative approaches have been developed to establish mixed chimerism. Conditioning of recipients with TBI plus cyclophosphamide (day +2) allowed mixed chimerism to be established with as low as 500 cGy TBI. The addition of ALG to the regimen allowed the TBI dose to be reduced to as low as 200 cGy TBI, Interestingly, conditioning of recipients with 300 cGy TBI plus ALG alone allowed chimerism to be established, but it was not durable.

In studies that defined the threshold dose of total body irradiation required for engraftment of syngeneic versus allogeneic marrow, it has been suggested that conditioning may be targeting specific host effector cells with differing radiation sensitivities. The invention presented herein uses compositions that target and specifically deplete effector cells in lieu of, or in combination with lower doses of, irradiation. According to the present invention, the targeting of specific effector cells results in a reduced requirement for TBI. More particularly, the present invention specifically depletes CD8⁺ and αβ-TCR⁺ cells by various pre-conditioning methods.

As previously discussed in detail, solid organ transplantation is currently dependent upon the use of nonspecific immunosuppressive agents to control rejection. The toxicities associated with the use of these immunosuppressive agents include infection, an increased frequency of malignancies, and end-organ toxicity. A major goal of research in transplantation has been to induce donor-specific transplantation tolerance and achieve permanent graft survival free from chronic nonspecific immunosuppressive agents. The present invention has attained this goal by establishing safe methods for inducing donor specific transplantation tolerance, thereby avoiding the expense and toxicity of immunosuppressive agents. Thus, the present invention provides for methods for engraftment using minimal conditioning strategies, thereby bringing tolerance closer to widespread clinical application.

In the present invention, anti-CD8 pretreatment was combined with anti-αβ-TCR to further reduce the TBI dose for conditioning with the rationale that specific host cellular subsets controlled the hematopoietic microenvironment for HSC engraftment. The improvements provided by the methods disclosed by the present invention draw from the discovery that a combination of anti-CD8 mAb and anti-αβ-TCR mAb is more potent than either antibody alone. However, the present invention is not limited to the use of antibodies to specifically deplete the CD8⁺ and αβ-TCR⁺ cells. As would be understood by those skilled in the art, the composition that specifically depletes the CD8⁺ and αβ-TCR⁺ cells may comprise antibodies specific for such cells, antisense DNA that is directed against the precursors of, or alters the transcription or translation of, those cells, or through the use of cytotoxic drugs that are specific for those cells.

This increased effectiveness of the present invention is that a combination of anti-Cb8 mAb and anti-αβ-TCR mAb is more potent than either antibody alone. This increased effectiveness could be due to one of two possible mechanisms. One explanation for this could be residual cell populations remaining after each mAb is administered. The presence of immunocompetent residual cells after mAb treatment that can mediate allorejection has been previously reported (Ichikawa, et al., Transplant. Proc., 19:579 (1987)); (Rosenberg, et al., J. Exp. Med., 173:1463 (1991)). Rosenberg, et al., reported that there was a small number of residual CD8⁺ cells left in recipient animals despite the fact that more than 99% of the CD8⁺ cells had been removed, and further, that these residual cells mediated the rejection of allogeneic skin grafts (Rosenberg, et al., J. Exp. Med., 173:1463 (1991)). When 2 mAbs with overlapping specificities cells were administered, more residual alloreactive cells were targeted. Therefore, the present invention, which shows that the combination of anti-αβ-TCR and anti-CD8 is more effective in establishing allogeneic chimerism than with a single mAb, is in agreement with these reports. A second possibility is that more than one host cell type contributes to alloresistance and that in addition to αβ-TCR⁺ T cells, a second CD8⁺ TCR⁻ cell serves as an effector cell as well.

It was surprisingly observed that the early production of donor T cells in the partially conditioned host is an absolute prerequisite for maintenance of mixed chimerism and induction of allograft tolerance. When recipients were preconditioned with the anti-αβ-TCR plus anti-CD8 mAb followed by 200 cGy TBI, chimerism occurred at relatively high levels but donor-derived T cells were not produced. When the TBI dose was increased to 300 cGy, 83.3% chimeras exhibited stable, long-term multilineage engraftment including production of donor T cells. It is of note that production of B cells, NK cells, granulocytes, and dendritic cells did not correlate with the durability of chimerism or induction of tolerance. These data confirm that early donor T cell chimerism is critical to achieve durable engraftment. One could hypothesize that a critical cell population in the host with a radiation sensitivity at 200-300 cGy TBI is responsible for this dichotomy or that cellular crosstalk between host and donor HSC or progenitors determines whether tolerance is induced at this threshold level for conditioning.

While a strict correlation between HSC chimerism and tolerance has historically been demonstrated in ablated recipients, recent reports have challenged the relationship between chimerism and tolerance, especially in partially conditioned and immunosuppressed recipients. As previously discussed, it has been suggested and observed that there is a dissociation between chimerism and allogenic tolerance in both microchimerism and macrochimerism models. Tolerance induction through chimerism in adults is hypothesized to involve multiple mechanisms including clonal deletion, anergy, and suppression or regulatory T cells. Clonal deletion, or negative selection, of alloantigen reactive cells is a major mechanism to achieve donor-specific tolerance to organ grafts. Clonal deletion can occur intrathymically (central deletion, central tolerance), in which the interaction between the immature T cell and thymic antigen-presenting cell leads to cell death, as well as peripherally (peripheral deletion) when activated T cells upregulate the expression of the surface Fas and FasL, which leads to their destruction by apoptosis.

In order to determine the mechanism required for the early events occurring in the induction of tolerance in the present model, it was evaluated whether donor T cell engraftment is critical to induce clonal deletion of graft-reactive cells. In chimeras with donor T cell chimerism, deletion of Vβ 5.1/2⁺ or Vβ 11⁺T cells occurred in both CD8 and CD4 T cells as expected. Chimeras without donor T cell engraftment showed no reduction in the percentage of Vβ 5.1/2⁺ or Vβ 11⁺ CD8 cells and a relatively lower reduction on the percentage of Vβ 5.1/2⁺ or Vβ 11⁺ CD4 cells compared to chimeras with donor T cell engraftment. Therefore, the ability to effect clonal deletion was directly correlated with production of donor T cells and not due to the presence of superantigen alone, nor did it correlate with the presence of donor dendritic cells.

Thus, one aspect of the present invention discloses that early donor T cell engraftment is associated with clonal deletion of donor-reactive T cells, as well as the maintenance of durable engraftment of MHC-disparate, allogeneic hematopoietic stem cells after transplantation. It is only in this context that chimerism is associated with functional tolerance.

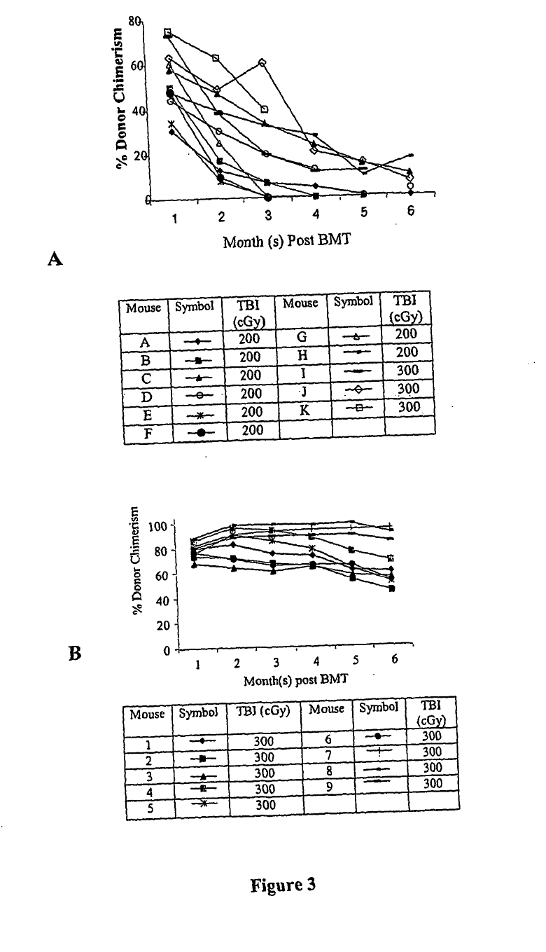

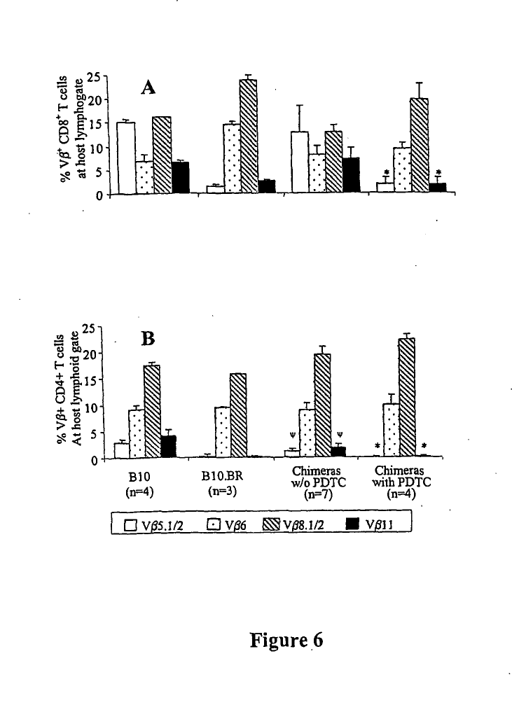

The present invention also evaluates whether donor T cells are produced but deleted in the thymus during T cell maturation. To evaluate early T cell development in chimeras with or without donor T cells, CD4⁻/CD8 thymocytes were stained and analyzed for CD24 expression as a marker of T lineage commitment. Surprisingly, donor CD24⁺ T cells were not detected in early stage pre-T cells (CD4⁻/CD8⁻) in chimeras that did not produce donor T cells. In contrast, donor CD24⁺/CD4⁻/CD8⁻ cells were present in thymus in chimeras with donor T cell production. The donor pre-T cells from chimeras with donor T cells in PB could be detected in the thymus at all stages of T cell maturation, from the most immature CD4⁻/CD8⁻/CD24⁺/CD44⁺/CD25⁻ to the mature single positive CD4⁺/8⁻ or CD4⁻/8⁺ cells (data not shown). From these data, it is known that the block in T cell development occurs at an extrathymic, very early, stage in maturation. The lack of mature donor T cells in PB and even very early stage of Pre-T cells in thymus might be due to the fact that: 1) donor stem cells are deficient in production of cells of T cell lineage; 2) donor-derived T lymphoid progenitors do not migrate to the recipient thymus; or 3) donor T lymphoid progenitors migrate to the thymus but are blocked very early prior to commitment to the T lineage. Taken together these data demonstrate a strong correlation between donor T cell maturation in the thymus and the induction of tolerance, most likely by clonal deletion. Taken together, one must hypothesize that no maturation of donor T cells in the thymus and no negative selection occurs on the donor-specific antigen present in the thymus in animals without T cell production. This would explain: 1) the lack of Vβ selection described in FIG. 6; 2) the lack of donor-specific tolerance in vivo and in vitro, and 3) the transient nature of the chimerism in these BMT recipients (FIG. 3A).

These findings also suggest that the components of donor chimerism may be ancillary phenomena, but not necessarily the mechanism for tolerance induction. It is more important to know what mechanisms, such as clonal deletion, are behind tolerance induction through chimerism. Thus, donor specific tolerance could occur if the clonal deletion is initiated in the host, regardless of the type of conditioning used or which donor cell populations engrafted. This could explain why different donor components are observed in tolerant chimeras in the radiation-based vs. non-radiation-based tolerance models. Tolerance could instead depend on the components that allow induction of host clonal deletion, which may be influenced by the conditioning approach used as well as the types of donor lineage cells present in the host. It is possible that confirming evidence for an active process for clonal deletion may be a more reliable predictor of clinical tolerance to organ allografts that the presence of donor chimerism.

In light of the fact that production of donor T cells was absolutely correlated with functional donor-specific tolerance in vivo and in vitro, as well as efficient clonal deletion, the discovery presented herein emphasizes the importance of donor T lineage-specific chimerism in the maintenance of stable mixed chimerism and donor-specific tolerance after nonmyeloablative allogeneic BM transplantation. This discovery provides indirect evidence that clonal deletion is the most likely mechanism for tolerance induction in the BMC transplantation model presented herein. A clear definition of the requirements that influence the induction of chimerism and tolerance will allow in vitro and clinically relevant in vivo strategies to potentiate the outcome to establish chimerism with minimal or no conditioning.

The invention is further illustrated by the following non-limited examples. All scientific and technical terms have the meanings as understood by one with ordinary skill in the art. The specific examples that follow illustrate the methods in which the present invention may be performed and are not to be construed as limiting the invention in sphere or scope. The methods may be adapted to variation in order to be embraced by this invention but not specifically disclosed. Further, variations of the methods to produce the same compositions in somewhat different fashion will be evident to one skilled in the art.

### EXAMPLES

### Materials and Methods

Animals. Male C57BL/10SnJ (B10, H-2^{b}), B10.BR/SgSnJ (B10.BR, H-2^{k}), and BALB/cJ (BALB/c, H-2^{d}) mice were purchased from the Jackson Laboratory (Bar Harbor, Me.). Animals were housed in the barrier facility at the Institute for Cellular Therapeutics and cared for according to National Institutes of Health animal care guidelines.

Assessment of in vivo depletion of CD8⁺ and αβ-TCR⁺ T cells and coating of γδ-TCR⁺ T cell. The mAbs anti-αβ-TCR(H57-597), anti-γδ-TCR (UC7-13D5) and anti-CD8 (53-6.7) were diluted in saline to 1 mL in previously titrated doses and injected intravenously through the lateral tail vein. The CD8 and αβ-TCR mAb are depleting while the γδ-TCR mAb is nondepleting. To document depletion, peripheral blood (PB) was obtained 3 days after mAb treatment from treated mice and stained with PE conjugated anti-αβ-TCR(H57-597), anti-γδ-TCR (GL3) and anti-CD8 (53-6.7). Staining was also performed with secondary mAbs of mouse anti-hamster IgG-PE or mouse anti-rat IgG2a-FITC to assure that cells were depleted or coated with mAbs. 100 μg was the dosage required to deplete CD8⁺ and αβ-TCR⁺, as well as to saturate γδ-TCR⁺ T cells in normal recipients. All mAbs used in vivo studies were produced and purified in house.

Chimera Preparation. Recipient B10 mice were pretreated intravenously with mAbs of anti-αβ-TCR (100 μg/each), anti-γδ-TCR (100 μg/each) and anti-CD8 (100 μg/each) alone or in combination 3 days before BMT. All mAbs used in vivo were produced and purified in house. On day 0, recipients were conditioned with 100, 200 or 300 cGy TBI (Gamma-cell 40, Nordion, Ontario, Canada). Animals were transplanted with 15×10⁶ untreated B10.BR bone marrow cells via lateral tail vein injection between 4 to 6 hours after irradiation. Each experiment was repeated at least three times.

Donor bone marrow was prepared by a modification of the method previously described {Colson, Wren, et al. 1995 308 /id} {Ildstad & Sachs 1984 711 /id}. Briefly, B10.BR donor mice were euthanized and tibias and femurs harvested. Bone marrow was expelled from the bones with Media 199 (GIBCO, Grand Island, N.Y.) containing 10 μg/mL Gentamicin (GIBCO), referred to hereafter as chimera medium (CM). The marrow was then gently prepared as a single cell suspension using a 3 cc syringe and 18-gauge needle. The cells were filtered through sterile nylon mesh with 100 μm pores, centrifuged at 1000 rpm for 10 minutes at 4° C., and resuspended in CM. A cell count was performed and the cells were diluted to a final concentration of 15×10⁶ bone marrow cells per mL of CM.

Characterization of chimeras. Recipients were characterized for chimerism using flow cytometry to determine the relative percentages of donor-derived peripheral blood lymphocytes (PBL) 1 month post BMT, and then monthly. Peripheral blood was obtained through tail vein bleeding and stained with antibodies specific for MHC Class I antigens of donor (PE conjugated anti-H2K^{k}, 36-7-5, mouse IgG₂ₐ) and recipient (FITC conjugated anti-H2 K^{b}, AF6-88.5, mouse IgG₂ₐ) origin. Briefly, 50 μL of whole blood was incubated with antibodies for 30 minutes at 4° C. in the dark. The blood was then incubated at room temperature for 6 minutes with ammonium chloride lysing buffer to eliminate erythrocytes and washed twice. The analysis was carried out on a FACS-Calibur (Becton Dickinson, Mountain View, Calif.) with CellQuest software (Becton Dickinson). Multi-lineage engraftment was assessed by four-color staining for FITC-conjugated anti-donor specific antibody (H2K^{k}) and different fluorescein (PE, PerCP and APC) conjugated lineage makers, including T cells (anti-CD4, RM4-5; anti-CD8×53-6.7; and anti-TCR-β, H57-597), B-cells (anti-B220, RA3-6B2), NK cells (anti-NK1.1, PK136), dendritic cells (anti-CD11c, HL3) and myeloid cells (anti-GR-1, RB6-8C5 and anti-MAC-1, M1/70). The following mAbs were utilized to analyze T cell development: anti-CD24 (30-F1), anti-CD25 (PC61) and anti-CD44 (IM7). Nonspecific background staining was controlled by using isotype control antibodies directed against irrelevant antigens conjugated with the same fluorochrome as the experimental antibody (i.e., anti-TNP mouse IgG2a mAb, conjugated with FITC, served as an isotype control for FITC-conjugated anti-H2 K^{b} mouse IgG2a). All mAb were obtained from PharMingen (San Diego, Calif.).

Flow cytometric analysis of TCR vβ families. Peripheral blood (80-100 μL) from unmanipulated control mice and mixed chimeras 1-6 months after reconstitution was stained with anti-Vβ 5.1/2-FITC (mr9-4), Vβ 6-FITC (rr4-7), Vβ 8.1/2-FITC (mr-5-2) or Vβ 11-FITC (rr3-15) vs. anti-host h2k^{b}-pe, anti-cd8-percp, and anti-cd4-apc (all from pharmingen) for 45 minutes at 4° C. A minimum of 50,000-gated events were collected within the total lymphoid gate the same day of staining. Samples were kept on ice prior to acquisition. Background staining was determined by FITC-conjugated isotype mAbs.

Skin Grafting. Skin grafting was performed by a modification of the method of Billingham {Billingham 1961 145 /id}. Full-thickness tail skin grafts were harvested from the tails of B10.BR(H2^{k}, donor-specific) and BALB/c (H2^{d}, third-party) mice. Recipient mice were anesthetized with Nembutal (pentobarbital sodium injection, Abbott, North Chicago, IL), and full-thickness graft beds were prepared surgically in the lateral thoracic wall, preserving the panniculus carnosum. The grafts were covered with a double layer of Vaseline gauze and a plaster cast. Casts were removed on the seventh day and grafts were scored by daily inspection for the first month and then weekly thereafter for percentage of rejection. Rejection was defined as complete when no residual viable graft could be detected.

One-way mixed lymphocyte reactions. MLR were performed as previously described {Hoffman, Langrehr, et al. 1990 679 /id}. Briefly, splenocytes were made into single cell suspensions, lysed free of RBCs, washed, and resuspended in DMEM supplemented with 5% FBS, 1 mM sodium pyruvate, 2 mM L-glutamine, 10 mM HEPES buffer solution, 0.137M L-Arginine HCL, 1.36 mM/0.027 M Folic Acid/L-Asparagine, 100 U/mL penicillin, 100 U/mL streptomycin (all from Gibco BRL), 0.05 mM 2-ME (Sigma). 2.5×10⁵ responder cells were cultured 1:1 with irradiated stimulator cells (2000 cGy) for 5 days at 37° C. in 5% CO₂. Each cell well was pulsed with 1 μCi [3H] thymidine (DuPont NEN, Boston, Mass.) 16 hr before harvesting with an automated harvester (PHD Cell Harvester Technology, Cambridge, Mass.).

Statistical Analysis. Data are presented as Average±Standard Deviation (SD). One tailed t-test (two sample assuming unequal variances) was used to evaluate statistical differences. The difference between groups was considered to be significant if P<0.05.

### Example 1

### Pretreatment of the Recipient with Anti-αβ-TCR Plus Anti-CD8 mAb Lowers the Requirement of TBI for Engraftment

It was previously demonstrated by the inventor that conditioning of mice with 700 cGy total body irradiation (TBI) was required to achieve engraftment of MHC-disparate allogeneic marrow in 100% of recipients {Colson, Wren, et al. 1995 308 /id}. Recipients exhibited durable chimerism and donor specific tolerance to skin and primarily vascularized cardiac allografts {Colson, Wren, et al. 1995 308 /id}. The TBI dose could be further reduced to 500 cGy if 200 mg/kg of intraperitoneal cyclophosphamide (CyP) was administered 2 days after allogeneic BMT {Colson, Wren, et al. 1995 308 /id}. The addition of anti-lymphocyte globulin (ALG) on day −3 reduced TBI dose to 300 cGy for allogeneic marrow engraftment {Colson, Li, et al. 1996 312 /id}. Replacement of ALG with in vivo administration of anti-CD8 mAb also resulted in engraftment at 300 cGy TBI {Exner, Colson, et al. 1997 437 /id}.

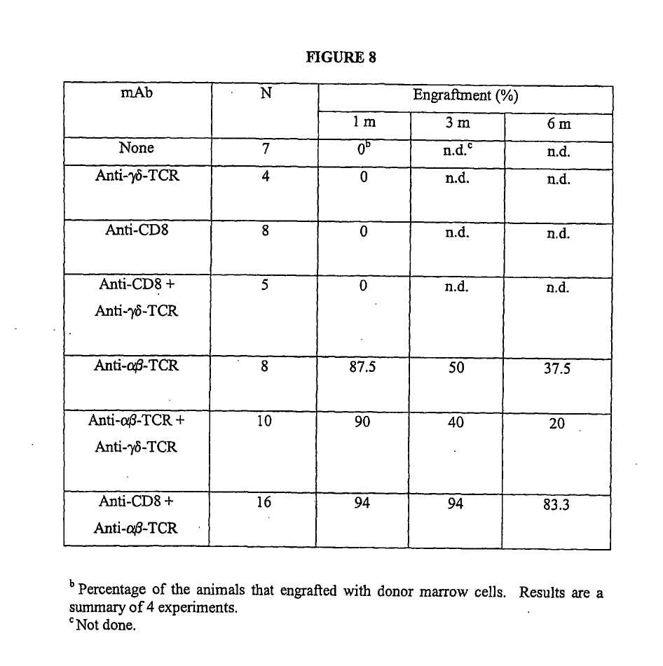

In this study the specific cell populations in the host which must be eliminated to enhance allogeneic BM engraftment (with the goal to eliminate the requirement for CyP and reduce the TBI dose as low as possible) were characterized. To evaluate the efficacy of targeting different T cell populations in allogeneic engraftment, recipient B10 mice were pretreated with anti-αβ-TCR, anti-γδ-TCR, anti-CD8, anti-αβ-TCR plus anti-γδ-TCR and anti-αβ-TCR plus anti-CD8 mAbs 3 days before BMT. On day 0, mice were conditioned with 300 cGy TBI and then transplanted with 15×10⁶ untreated bone marrow cells from B10.BR donors 4-6 hours later (See FIG. 8).

Animals were typed by flow cytometric analysis monthly for up to 6 months after BMT. With 300 cGy TBI, allogeneic engraftment did not occur in animals preconditioned by anti-CD8 alone, anti-γδ-TCR alone or anti-CD8 plus anti-γδ-TCR. In contrast, high levels of engraftment were established in animals preconditioned with anti-αβ-TCR alone, anti-αβ-TCR plus γδ-TCR and anti-αβ-TCR plus anti-CD8 (87.5%, 90% and 94%, respectively) 1 month post BMT. Interestingly, long-term engraftment (up to 6 months) was only achieved in mice pre-conditioned with both anti-αβ-TCR and anti-CD8. The fact that both antibodies in combination achieved the most durable engraftment in the highest proportion of recipients suggests that these agents may be targeting additional CD8⁺ cells in the recipient that are not αβ-TCR⁺.

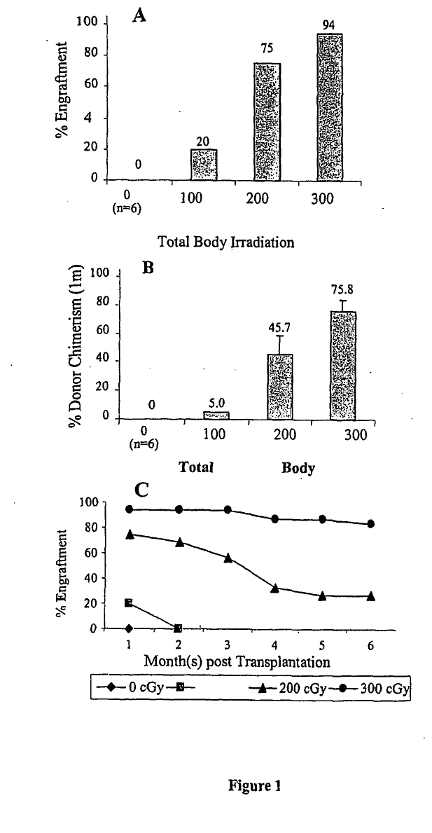

A dose-titration of TBI was performed to determine the minimal conditioning required with pre-conditioning with the anti-αβ-TCR plus anti-CD8 combination. B10 mice were pretreated with anti-αβ-4-TCR plus anti-CD8 on day −3 and transplanted with 15×10⁶ untreated bone marrow cells from B10.BR donors following conditioning with 0, 100, 200 or 300 cGy TBI. Ninety four percent of mice conditioned with 300 cGy (n=16) engrafted 1 month after BMT (FIG. 1A). Seventy five percent engrafted with 200 cGy (n=16) (FIG. 1A). Only 20% of mice engrafted when conditioned with 100 cGy (n=5) and none engrafted without TBI (n=6). The levels of donor chimerism were directly correlated with the amount of conditioning, at 75.8±7.7%, 45.7±12.6% and 5.0% one month post-BMT with 300, 200 and 100 cGy TBI, respectively (FIG. 1B). Engraftment remained high (83.3% at 6 months after BMT) in animals conditioned with 300 cGy TBI while the majority of engrafted animals (8 out of 12) lost their chimerism after conditioning with 200 cGy and all with 100 cGy TBI (FIG. 1C). These results suggest that the TBI dose can be reduced from 700 to 300 cGy by depletion of both host αβ-TCR⁺ and CD8⁺ T cells in vivo. Moreover, these results further confirmed the inventor's previous finding that both αβ-TCR⁺ and CD8⁺ T cells in the host play critical and nonredundant roles in preventing engraftment of allogeneic bone marrow {Xu, Exner, et al. 2002 3843 /id}.

### Example 2

Production of Donor T Cells is Critical for the Maintenance of Stable Mixed Chimerism

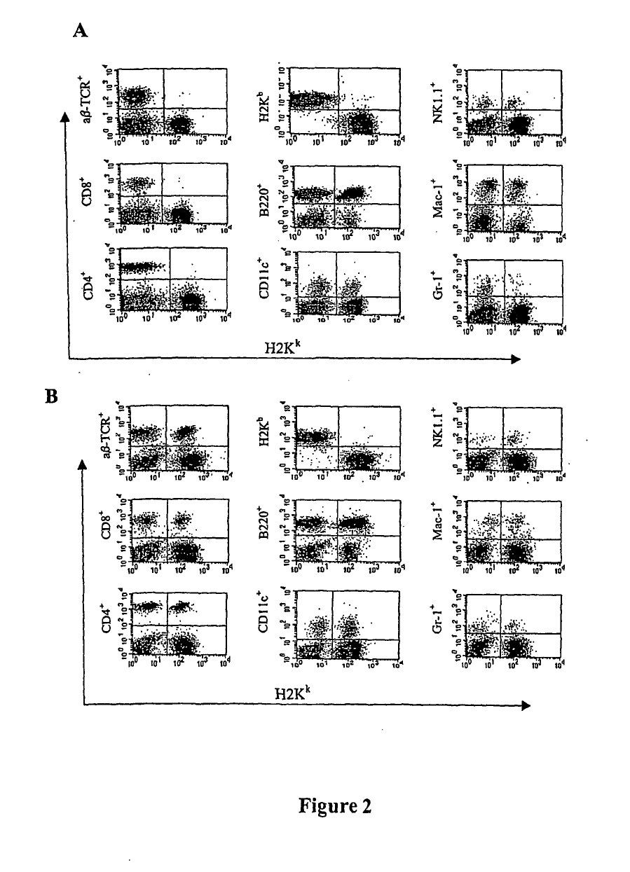

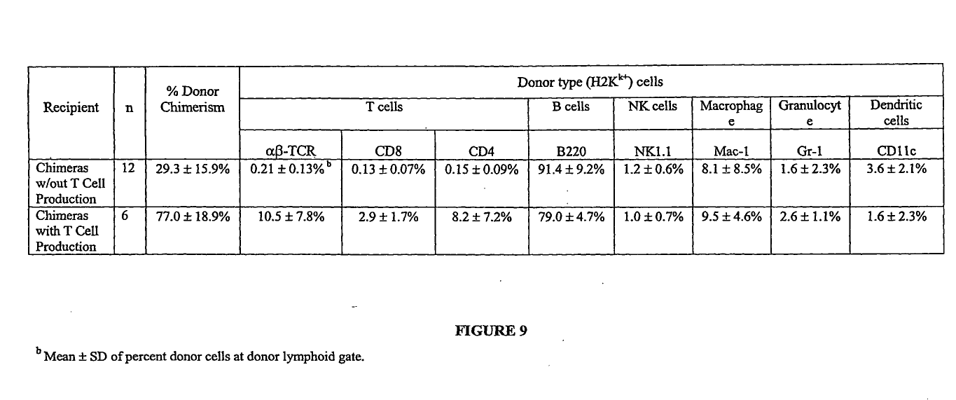

The pluripotent hematopoietic stem cell (HSC) produces at least 11 different lineages. To evaluate the influence of multilineage production on the durability of engraftment and induction of tolerance, animals were followed for ≧4 months by four-color flow cytometric analysis (donor versus lineage). In mixed chimeras conditioned with either 200 cGy or 300 cGy, a dichotomy for donor T cell engraftment that predicted both durability of engraftment and tolerance was found (FIG. 2). The chimeras were evaluated according to whether they produced donor T cells irrespective of conditioning. In Group A, although B cells, NK cells, monocytes and dendritic cells of donor origin were detected, no donor-derived αβ-TCR⁺, CD4⁺ or CD8⁺ T cells were present (See FIGS. 2A and 9). All chimeras conditioned with 200 cGy TBI (n=12) failed to produce donor T cells and 40% of chimeras (n=15) conditioned with 300 cGy failed to produce donor T cells (FIG. 3A). For the second phenotype (Group B, FIG. 2B), all lineages of donor origin were present. All of these chimeras had been conditioned with 300 cGy TBI. These phenotypes did not change during the time course that chimerism was evaluated.

Most recipients conditioned with 200 cGy TBI lost their chimerism within 6 months. In striking contrast, chimerism was durable in the majority (83.3%) of animals conditioned with 300 cGy TBI. The initial percentage chimerism ranged from 30.4% to 75.3% in this cohort. In animals with donor T cell production, mixed chimerism remained stable for ≧6 months (FIG. 3B). The level of chimerism was 82.7%±6.4% at 1 month post-transplantation in the group with donor T cell engraftment. At 3 months, donor chimerism was 87.9%±14.1% and remained stable (67.2%±18.8%) for ≧6 months. In striking contrast, animals without donor T cell production lost their chimerism gradually within 6 months (FIG. 3A). The level of chimerism significantly decreased over time in chimeric mice without donor T cell engraftment, although chimerism initially averaged 53.2%±14.4% at 1 month, and 30.7%±18.1% at 2 months post transplantation (P<0.005) in this group. These findings suggest that the production of donor T cells is critical for the maintenance of stable chimerism.

### Example 3

Production of Donor T Cells is Critical for Induction of Donor-Specific Tolerance to Skin Grafts

Skin grafts were performed to assess donor-specific tolerance in vivo in the two groups. Each animal received a skin graft from donor-specific (B10.BR) and third-party (BALB/c, H2^{d}) strains 2-3 months after BMT. Grafts were assessed daily for the first 4 weeks and weekly thereafter for evidence of rejection. Animals that failed to engraft donor stem cells at 1 month after transplantation rejected both donor and third-party grafts promptly (median survival time (MST)=10 days for both grafts). The chimeras without donor T cells rejected third party skin grafts in a fashion similar to that observed in mice without chimerism. Surprisingly, however, the majority of chimeras without donor T cells promptly rejected donor skin grafts as well (MST=12.5 days), with a time course similar to the third-party grafts, in spite of the presence of significant levels of donor chimerism at the time of graft placement. The level of donor chimerism in this group was 37.6±27.4% (range 7.1% to 72.7%) at the time the skin transplantation was performed. Only one of 18 (5.6%) donor skin grafts survived >120 days in this group. In chimeras with donor T cell engraftment, donor-specific allogeneic skin grafts were permanently accepted (MST>120 days) in 7 out of 9 mice and the survival of the other 2 grafts was prolonged, while third-party skin grafts were promptly rejected. Donor-specific skin graft survival in the group with donor T cell engraftment was significantly prolonged compared with the group without donor T cell production (P<0.00005). These data show that production of donor T cells is critical for the induction of donor-specific tolerance in nonmyeloablated-conditioned recipients.

### Example 4

Evidence for Donor-Specific Tolerance In Vitro in MLR Assay

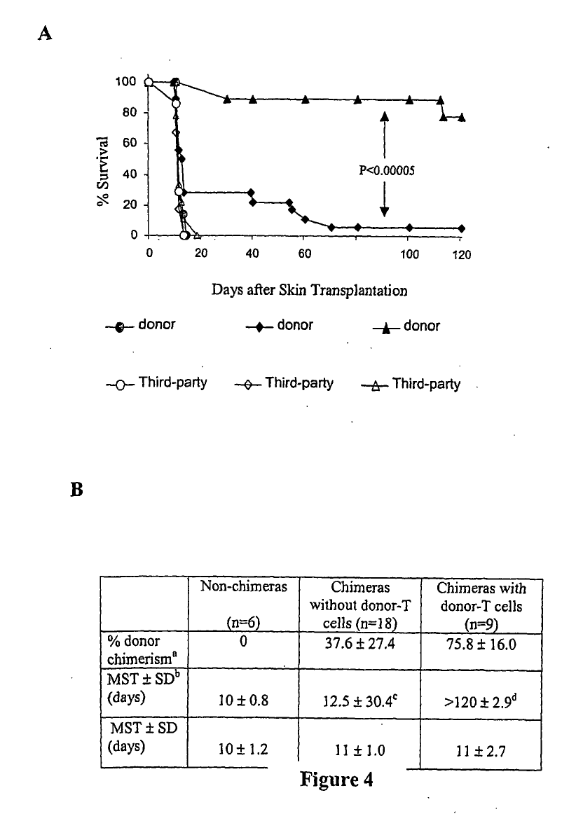

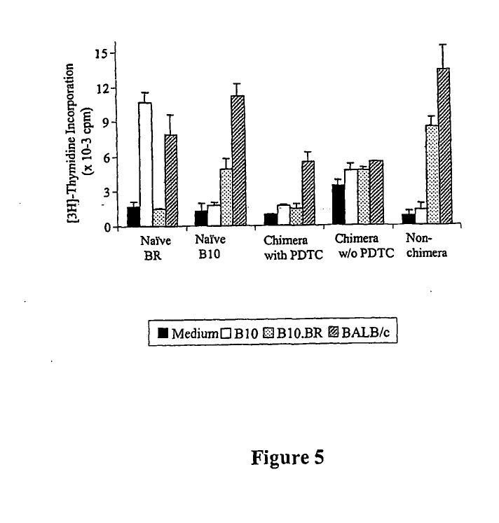

Splenic lymphoid cells from chimeras with (n=8) or without (n=6) donor T cell production, as well as from recipients that did not engraft (non-chimeras, n=7), were assessed for donor-specific tolerance in vitro using one-way MLR assays directed against host, donor and third-party irradiated stimulator cells. As seen in a representative one-way MLR assay (FIG. 5), chimeras with donor T cells exhibited tolerance to both host (B10) and donor strain (B10.BR) stimulators but were reactive to MHC-disparate third-party (BALB/c) stimulators. Non-chimeras were reactive to both donor and third-party stimulators but not reactive to host stimulators. Chimeras without donor T cells exhibited reactivity to donor and third-party stimulators. Moreover, proliferation in the presence of host stimulators and even in medium control wells was as high as in donor and third-party wells. These unexpected results can be explained by the fact that the spleens from these animals contained both donor and host cells that are not tolerant to each other. Therefore, with medium alone, the host T cells proliferate in response to the stimulation from the allogeneic donor cells of mixed chimeras. These in vitro data confirm the presence of specific tolerance to donor strain alloantigens in chimeras with peripheral donor T cells and absence of donor-specific tolerance in chimeras without peripheral donor T cells, consistent with the results of skin graft survival seen in these two groups of chimeras (FIG. 4).

### Example 5

Production of Donor T Cells is Critical for Clonal Deletion of Donor-Reactive TCR-Vβ Subsets

While not wishing to be bound by any theory, it was hypothesized that donor T cell engraftment may be critical to induce or regulate deletion of graft-reactive cells. To investigate whether clonal deletion is operational in the present model, the expression of superantigen-specific T cells, Vβ 5.1/2, Vβ 6, Vβ 8.1/2, and Vβ 11 in chimeras with or without donor T cell engraftment was measured. B10 and B10.BR splenocytes served as controls. Relative expression indicates the percentage of Vβ-positive cells within the CD8⁺ or CD4⁺ T cell subsets of the host (H2K^{b}) lymphoid gate in peripheral blood. The host lymphoid gate used in the current study for clonal deletion analysis is more accurate than the total (host+donor) lymphoid gate used previously {Wekerle, Kurtz, et al. 2001 3747 /id} {Wekerle, Sayegh, et al. 1998 2468 /id} {Colson, Lange, et al. 1996 314 /id} by avoiding an effect from the deleted donor Vβ 5.1/2⁺ and Vβ 11⁺ cells. The donor strain B10.BR mice express I-E, resulting in the deletion of Vβ 5.1/2⁺ and Vβ 11⁺ T cells {Abe, Kanagawa, et al. 1991 9 /id} {Tomonari & Fairchild 1991 4029 /id}. As B10 mice do not express I-E, they do not delete these two Vβ subfamilies {Tomonari & Fairchild 1991 4029 /id} {Bill, Kanagawa, et al. 1989 4030 /id}. Chimeras with donor T cell engraftment showed the same relative Vβ expression as B10.BR mice, suggesting the deletion of Vβ 5.1/2⁺ and Vβ 11⁺ subfamilies of both CD4⁺ and CD8⁺ T cells (FIGS. 5A and 5B, P<0.005). This negative selection was specific, as Vβ 6 and Vβ 8.1/2 subsets were not deleted. Chimeras without donor T cells exhibited Vβ expression similar to recipient B10 mice in CD8⁺ T cells, indicating that no deletion of Vβ 5.1/2⁺ and Vβ 11⁺ subfamilies of CD8⁺ T cells occurred (FIG. 5A). In addition, only partial deletion of Vβ 5.1/2⁺ and Vβ 11 of CD4⁺ T cells was observed in chimeras without donor T cells (FIG. 5B). The level of Vβ 5.1/2⁺ and Vβ 11⁺ CD4 T cells in chimeras without donor T cells was significantly higher than in chimeras with donor T cell production (P<0.005). However, the reduced level of Vβ 5.1/2⁺ and Vβ 11⁺ CD4⁺ T cells in chimeras without donor T cells was statistically significant compared with control B10 mice (FIG. 5B, P<0.05), the levels of Vβ 5.1/2⁺ and Vβ 11⁺ of CD4 T cells in chimeras without donor T cells suggesting that partial deletion had occurred. At the time of analysis, animals still had significant levels of donor chimerism in chimeras with (68.9%±15.8%) or without (48.9%±19.3%) donor T cell engraftment.

### Example 6

Early Stage Pre-T Cells (CD24⁺/CD4⁻/CD8⁻) are not Present in the Thymus of Chimeras without Peripheral Donor T Cells

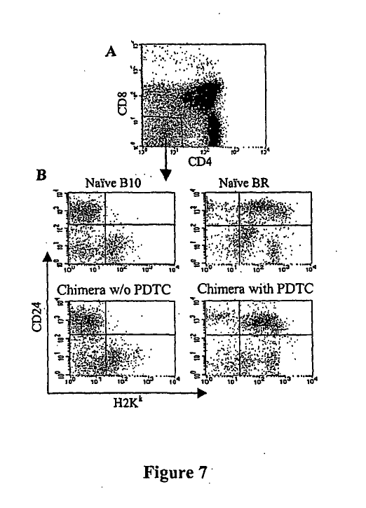

Next evaluated was where the block in T cell development was taking place. CD24 (heat-stable antigen [HAS]) is a marker of T lineage commitment in early stage of T cell development (CD4⁻/CD8⁻) in thymus {Ceredig & Rolink 2002 1996 /id}. It is expressed at high levels during the double negative (DN) stage {Ge & Chen 1999 1995 /id}. In order to determine if pre-T cells were present in thymus without PDTC, thymocytes were analyzed by flow cytometry. A single cell thymocyte suspension was prepared from chimeras with or without donor T cells in PB and from controls of naïve B10 or B10.BR mice. Cells were stained with CD4 APC, CD8 PE, CD24 PerCP and donor class I H2K^{k} FITC. As shown in FIG. 7, donor CD24⁺ T cells were not detected in the double negative (CD4⁻/8⁻) thymocytes in chimeras devoid of donor peripheral T cells. Their staining pattern is identical to that of naïve recipient B10 mice. In contrast, donor CD24⁺/4⁻/8⁻ thymocytes were present in chimeras with donor peripheral T cells. The staining pattern of these chimera strongly resembled that of the naïve donor strain, B10.BR.

The foregoing description is considered as illustrative only of the principles of the invention. Further, since numerous modifications and changes will readily occur to those skilled in the art, it is not desired to limit the invention to the exact construction and process shown as described above. Accordingly, all suitable modifications and equivalents may be resorted to falling within the scope of the invention as defined by the claims that follow.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

The embodiments of the invention in which an exclusive property or privilege is claimed are defined as follows:

1. A method for conditioning a recipient for bone marrow transplantation comprising subjecting said recipient to a composition that specifically depletes αβ-TCR⁺ T cells and CD8⁺ T cells in the recipient hematopoietic microenvironment, followed by transplantation with a donor cell preparation containing hematopoietic stem cells from a donor that are matched at the major histocompatibility complex class I K locus with the recipient hematopoietic microenvironment.

2. The method of claim 1 in which said composition comprises antibodies specific for αβ-TCR⁺ T cells and CD8⁺ T cells.

3. The method of claim 1 in which said composition comprises antisense DNA that is directed against the precursors of αβ-TCR⁺ T cells and CD8⁺ T cells.

4. The method of claim 3 wherein antisense DNA alters the translation of the α-chain or β-chain of TCR⁺ T cells.

5. The method of claim 3 wherein antisense DNA alters the transcription of the α-chain or β-chain of TCR⁺ T cells.

6. The method of claim 1 in which said composition a cytotoxic drug specific for αβ-TCR⁺ T cells and CD8⁺ T cells.

7. The method of claim 1 wherein the recipient is further conditioned by subjecting the recipient to a total dose of total body irradiation of less than or equal to 300 cGy.

8. The method of claim 1 wherein the recipient is further conditioned by subjecting the recipient to an alkylating agent.

9. The method of claim 8 wherein said alkylating agent is cyclophosphamide.

10. The method of claim 1 wherein said composition specific to αβ-TCR⁺ T cells and CD8⁺ T cells in the recipient hematopoietic microenvironment totally eliminates said cells from the recipient hematopoietic microenvironment.

11. A method for conditioning a recipient for bone marrow transplantation comprising subjecting said recipient treatment with a total dose of total body irradiation from 100 to 300 cGy, and treating the patient with a composition that specifically depletes (αβ-, TCR⁺ T cells and CD8⁺ T cells in the recipient hematopoietic microenvironment, followed by transplantation with a donor cell preparation containing hematopoietic stem cells from a donor that are matched at the major histocompatibility complex class I K locus with the recipient hematopoietic microenvironment.

12. The method of claim 11 wherein the recipient is further treated with an alkylating agent before, during, or after exposure to said composition that specifically depletes αβ-TCR⁺ T cells and CD8⁺ T cells in the recipient hematopoietic microenvironment.

13. The method of claim 12 wherein said alkylating agent is cyclophosphamide.

14. A method of partially or completely reconstituting a mammal's lymphohematopoietic system comprising administering to the mammal a composition that specifically depletes αβ-TCR⁺ T cells and/or CD8⁺ T cells in the recipient hematopoietic microenvironment, followed by transplantation with a donor cell preparation containing hematopoietic stem cells from a donor that are matched at the major histocompatibility complex class I K locus with the recipient hematopoietic microenvironment.

15. The method of claim 14, in which the mammal suffers from autoimmunity.

16. The method of claim 15 in which the autoimmunity is diabetes.

17. The method of claim 15, in which the autoimmunity is multiple sclerosis.

18. The method of claim 15, in which the autoimmunity is sickle cell.

19. The method of claim 15, in which the autoimmunity is anemia.

20. The method of claim 15, in which the mammal suffers from a hematologic malignancy.

21. The method of claim 14, in which the mammal requires a solid organ or cellular transplant.

22. The method of claim 14, in which the mammal suffers from immunodeficiency.
